(19) United States
(12) Patent Application Publication
(10) Pub. No.: US 2025/0241563 A1
(43) Pub. Date: Jul. 31, 2025

(54) METHOD, SYSTEM, AND PRODUCT FOR OXIMETRY DEVICE FOR IMPROVING MEASUREMENT QUALITY

(71) Applicants: Temple University-Of The Commonwealth System of Higher Education, Philadelphia, PA (US); Washington University in St. Louis, St. Louis, MO (US)

(72) Inventors: Chetan Patil, Haddonfield, NJ (US); Mohammed Shahriar Arefin, Philadelphia, PA (US); Leo Shmuylovich, St. Loius, MO (US)

(21) Appl. No.: 19/036,632

(22) Filed: Jan. 24, 2025

Related U.S. Application Data
(60) Provisional application No. 63/625,154, filed on Jan. 25, 2024.

(51) Int. Cl. A61B 5/1455

(52) U.S. Cl. CPC A61B 5/14551

(57) ABSTRACT

A device for measuring blood oxygen saturation including at least two emitters. The device comprises an IR emitter configured to emit a first signal at a first wavelength. This signal is able to be absorbed in part by oxyhemoglobin. The device may also include a second emitter to emit at a separate second wavelength. This wavelength is able to be absorbed in part in by deoxyhemoglobin. The device may also include one or more detectors configured waveforms corresponding to both the signals from the emitters. These waveforms may comprise AC and DC components which are to be used by the processor(s) of the device. The processor(s) is configured to calculate an oximeter ratio (R) based on the respective AC and DC components.

### CROSS-REFERENCE TO RELATED APPLICATIONS

This application claims priority to U.S. Provisional Patent Application No. 63/625,154, filed on Jan. 25, 2024, incorporated herein by reference in its entirety.

### BACKGROUND OF THE INVENTION

Pulse oximeters are widely used clinical devices that provide optical estimates of blood oxygen saturation (see Chan, E. D. et al., Respir. Medicine). Although, it has been widely used throughout the healthcare systems, there have been reports of erroneous readings from pulse oximetry (see Ralston, A. C. et al., Anaesthesia, 1991) (see Fine, J. et al., Biosensors, 2021; Jubran, A., Critical Care, 2015; and Ries, A. L. et al., 1989. CHEST). Recent retrospective studies have shown that pulse oximeters overestimate oxygen saturation in darkly pigmented racial minorities, which results in higher rate of occult hypoxemia and delays in care (see Sjoding, M. W. et al., 2020. New England Journal of Medicine; Fawzy, A. et al., JAMA Intern. Medicine, 2022; and Valbuena, V. S. M. et al., 2022, BMJ). Similarly, in silico Monte Carlo (MC) simulations have indicated that the oximeter ratio, R, which is used to estimate blood oxygen saturation level (SpO₂), carries a pigmentation dependence, which likely contributes to reports of racial bias (see Boonya-ananta, T. et al., Sci. Reports, 2021). Another MC study reported that calibration enrollment could play a role in this over-estimation bias phenomenon (see Arefin, M. S. et al., In Hwang, J. & Vargas, G. (eds.) Design and Quality for Biomedical Technologies, 2022). These reports suggest that there could be multiple sources which could introduce bias in oximetry.

Possible strategies to reduce pigmentation bias include modification of the device's optical design, pursuit of more equitable enrollment in calibration and testing trials, and modification of the algorithm which estimates SpO₂ (see Arefin, M. S. et al., In Hwang, J. & Vargas, G. (eds.) Design and Quality for Biomedical Technologies, 2022; NIH 2022; Baek, H. J. et al., 2018., Journal of Healthcare Engineering; Okunlola, O. E. et al., 2022. Respiratory Care; and Keller, M. D. et al., 2022. Nature). Previous study showed that optical design modification could reduce over-estimation bias in subjects with African ethnicity (see Baek, H. J. et al., 2018. Journal of Healthcare Engineering). An MC simulation study showed that inclusion of more highly pigmented subjects in the calibration enrollment could help to reduce over-estimation bias in highly pigmented subjects (see Arefin, M. S. et al., In Hwang, J. & Vargas, G. (eds.) Design and Quality for Biomedical Technologies XV, 2022). There are a number of commercially available ‘Skin Colorimeters’ and ‘Melanometers’ that perform objective spectrometric measurements of skin color and estimate melanin content with a dimensionless parameter called the Melanin Index (MI). The melanin index provides information related to the melanin content of the skin, which is the dominant chromophore in determining skin pigmentation.

However, introducing additional hardware, such as a Melanometer, to the pulse oximeter to correct biases increases total costs and overall size. Therefore, there is a need in the art for a lower cost, lighter, and straightforward solution by using simpler hardware configurations.

### SUMMARY OF THE INVENTION

Some embodiments of the invention disclosed herein are set forth below, and any combination of these embodiments (or portions thereof) may be made to define another embodiment.

In one aspect of the invention, a device for measuring blood oxygen saturation may include at least two emitters comprising one or more IR emitters configured to emit one or more first signals at one or more respective first wavelengths, wherein the one or more first signals are able to be absorbed in part by oxyhemoglobin; one or more second emitters configured to: emit one or more second signals at one or more respective second wavelengths, wherein the one or more second signals are able to be absorbed in part in by deoxyhemoglobin; one or more detectors configured to receive (i) one or more first waveforms corresponding to the one or more first signals and (ii) one or more second waveforms corresponding to the one or more second signals, wherein the one or more first and second waveforms each comprise AC and DC components; and one or more processors operatively coupled to the one or more detectors, wherein the processor is configured to calculate an oximeter ratio (R) based on the respective AC and DC components of the one or more first and second waveforms.

In one aspect of the invention, one or more IR emitters may emit one or more first wavelengths from about 850 nm to 1000 nm. In one aspect of the invention, the one or more second emitters may emit the one or more second wavelengths from about 600 nm to 750 nm. In another aspect of the invention, the one or more second emitters may emit the second wavelength from about 632 nm to 660 nm.

In one aspect of the invention, one or more processors may calculate the oximeter ratio to correct for one or more of:lipid content, hydration status, non-pulsatile tissue, chromophores, melanin, and venous blood volume, or any combination thereof. In one aspect of the invention, the device is capable of being placed on a forehead, nose, foot, ear, or toe of a patient.

In one aspect of the invention, the one or more IR emitters exclude additional IR emitters and consist of only one IR emitter to emit one first signal at one first wavelength, wherein the one first signal is able to be absorbed in part by oxyhemoglobin. In one aspect of the invention, the one or more second emitters exclude additional emitters, which may be in the optical spectra, and consist of only one second emitter to emit one second signal at one second wavelength, wherein the one second signal is able to be absorbed in part by deoxyhemoglobin.

In one aspect of the invention, the one or more IR emitters of the device consist of one IR emitter configured to emit a plurality of first signals at a respective plurality of first wavelengths; and wherein the one or more second emitters consist of one second emitter configured to emit a plurality of second signals at a respective plurality of second wavelengths.

In one aspect of the invention, the one or more IR emitters consist of one IR emitter to emit one first signal at one first wavelength, wherein the one first signal is able to be absorbed in part by oxyhemoglobin; and wherein the one or more second emitters consist of one second emitter to emit one second signal at one second wavelength, wherein the one second signal is able to be absorbed in part by deoxyhemoglobin; wherein the one or more first and second waveforms consist of one first waveform and one second waveform; one or more detectors are configured to receive (i) the one first waveform corresponding to the one first signal and (ii) the one second wave form corresponding to the one second signal, wherein the one first waveform and one second waveform each comprise AC and DC components, respectively; and wherein the calculated oximeter ratio (R) is based on the respective AC and DC components of the one first waveform and one second waveforms.

In one aspect of the invention, the oximeter ratio (R) is modified to correct for melanin using a calculated index (MI) to create a modified oximeter ratio (R). In one aspect of the invention, the calculated index (MI) is based on the respective DC components of the one first waveform and one second waveform. In one aspect of the invention, the calculated melanin index (MI) is based on a logarithmic function. In one aspect of the invention, the calculated index is used to account for blood volume. In one aspect of the invention, the oximeter ratio (R) is modified to correct for melanin using a calculated melanin index (MI) to create a modified oximeter ratio (R). In one aspect of the invention, the calculated melanin index (MI) is based on the respective DC components of the one first waveform and one second waveform. In one aspect of the invention, the calculated melanin index (MI) is based on a logarithmic function. In one aspect of the invention, the calculated melanin index is used to account for melanin. In one aspect of the invention, the one or more detectors comprise one or more photodetectors.

In one aspect of the invention, a device for measuring blood oxygen saturation comprising: one first emitter configured to: emit more than one first signal at more than one respective first wavelengths, wherein the more than one first signal is able to be absorbed in part by oxyhemoglobin; one second emitter configured to: emit more than one second signal at more than one respective second wavelengths, wherein the more than one second signal is able to be absorbed in part in by deoxyhemoglobin; one or more detectors configured to receive (i) more than one first waveforms corresponding to the more than one first signal and (ii) more than one second wave forms corresponding to the or more than one second signal, wherein the more than one first and second waveforms each comprise AC and DC components; and a processor operatively coupled to the one or more detectors, wherein the processor is configured to calculate a modified oximeter ratio (R) to improve measurement quality based on the respective AC and DC components of the more than one first and second waveforms.

In one aspect of the invention, a device for measuring blood oxygen saturation using only a first and second emitter comprising: the first emitter configured to emit a first signal at a first wavelength; the second emitter configured to a second signal at a second wavelength; one or more detectors configured to receive a first and second waveform corresponding to the first and second signal, respectively, wherein the first and second waveforms each comprise AC and DC components; and one or more processors operatively coupled to the one or more detectors, wherein the processor is configured to calculate a modified oximeter ratio (R) based on the AC and DC components of the first and second signals, wherein R is pigmentation independent.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1 depicts calibration and testing of oximeter parameters using MIₒₓᵢₘₑₜₑᵣ and R_{mod}. Calibration was generated from Monte Carlo simulations of oximeter calibration studies and were tested against test cohort to estimate SpO₂ and then compared against known oxygen saturation values to generate measures of bias and variance.

FIG. 2A and FIG. 2B depict error in test subjects with different fractional volume melanosome (FVM) showing over-estimation bias in high pigmentation level in the case of calibration constructed from 80:20 low:high melanin population shown in FIG. 2A and 50:50 low:high melanin population shown in FIG. 2B.

FIG. 3A and FIG. 3B depict error in test subjects with different FVM showing less over-estimation bias in high pigmentation level when R_{mod} is used instead of R. This less over-estimation is valid for calibrations constructed from 80:20 low:high melanin population as shown in FIG. 3A and 50:50 low:high melanin population as shown in FIG. 3B.

FIG. 4A depicts an aspect of the invention showing exemplary wavelengths and corresponding absorption coefficients of oxyhemoglobin, deoxyhemoglobin, and melanin at 660 nm and 940 nm. FIG. 4B depicts an aspect of the invention showing waveforms corresponding to AC and DC components based on the cardiac cycle. FIG. 4C depicts an aspect of the invention showing the calculation of the modulation ratio (R).

FIG. 5 depicts an aspect of the invention showing a calibration model.

FIG. 6A depicts an aspect of the invention showing a finger model with the source or emitter and the detector of an oximeter. FIG. 6B depicts an aspect of the invention of calibration equation development based on the finger model.

FIG. 7 depicts an aspect of the invention showing that oximeter ratio (R) is dependent on pigmentation.

FIG. 8 depicts an aspect of the invention on an exemplary piece of hardware of a Melanometer.

FIG. 9 depicts an aspect of the invention showing MI being calculated based on DC components of emitters which are a function of the fractional value of melanosome.

FIG. 10 depicts an aspect of the invention showing the pigmentation independence of R modified based on MI.

FIG. 11A depicts an aspect of the invention showing the result of R against R modified.

FIG. 12 depicts an aspect of the invention of an in silico application of modified ratio.

FIG. 13A depicts an aspect of the invention showing diagnostic accuracy of modified ratio R in simulated ICU patients for hypoxemia detection. FIG. 13B depicts an aspect of the invention showing tabulated information that a modified ratio R improves diagnostic accuracy using simulated patients.

FIG. 14 depicts an exemplary computing device in which aspects of the invention may be practiced.

### DETAILED DESCRIPTION

It is to be understood that the figures and descriptions of the present invention have been simplified to illustrate elements that are relevant for a clearer comprehension of the present invention, while eliminating, for the purpose of clarity, many other elements. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Ranges: throughout this disclosure, various aspects of the invention can be presented in a range format. It should be understood that the description in range format is merely for convenience and brevity and should not be construed as an inflexible limitation on the scope of the invention. Where appropriate, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The aforementioned systems, processes and methods described herein may be utilized for desired applications as would be appreciated by those skilled in the art. In one aspect, the present invention solves many of the issues of adding hardware such as additional emitters and/or detectors to correct for biases in certain patients. For example, there may be only one emitter within the optical band that emits redlight, and/or there may be only one emitter that emits within the IR band. In another aspect, the invention solves issues associated with adding Melanometers and similar hardware to account for biases based on melanin in subjects that may have darker pigmentation. For example, instead of using a Melanometer, an oximeter ratio may be modified to be pigmentation independent in order to reduce the over-estimate in individuals with darker pigmentation. In another aspect, the present invention solves issues with biases in subjects by using a plurality of emitters.

Oximeters are electro-optical devices with embedded electronics and processor(s) that perform calculations to calculate blood oxygen saturation. In one embodiment, the pulse oximeter may be shaped or configured to be placed on the fingers preferably. In various alternative embodiments, the pulse oximeter may be shaped or configured to be placed on the forehead, nose, foot, ears, or toes.

In one embodiment, the pulse oximeter may include one or more emitters. In another embodiment, the pulse oximeter may only include one emitter within the optical band emitting, for example, redlight and/or only include another emitter emitting with the IR band. For example, there may only be two emitters included such that only one is within the optical band (e.g., redlight) and only one is within the IR band.

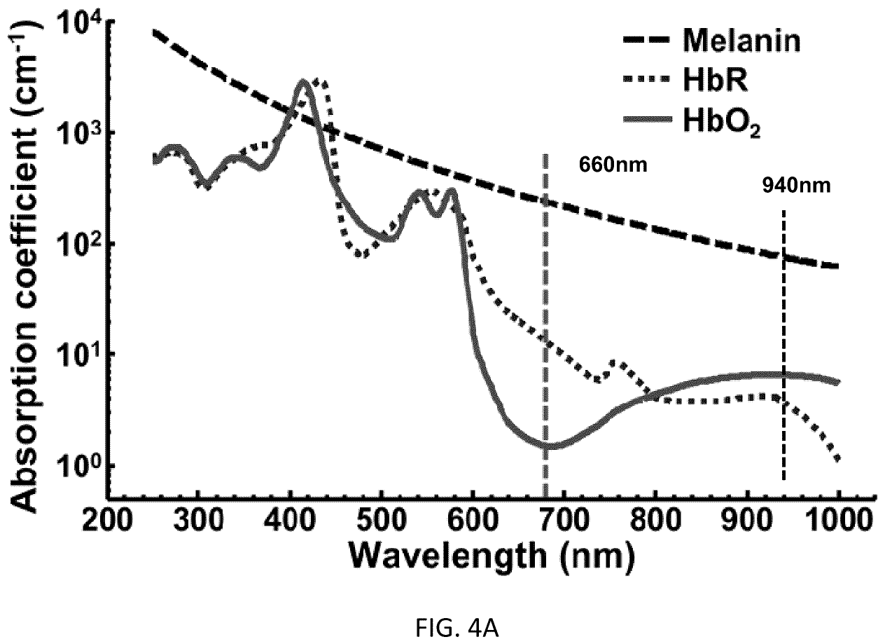

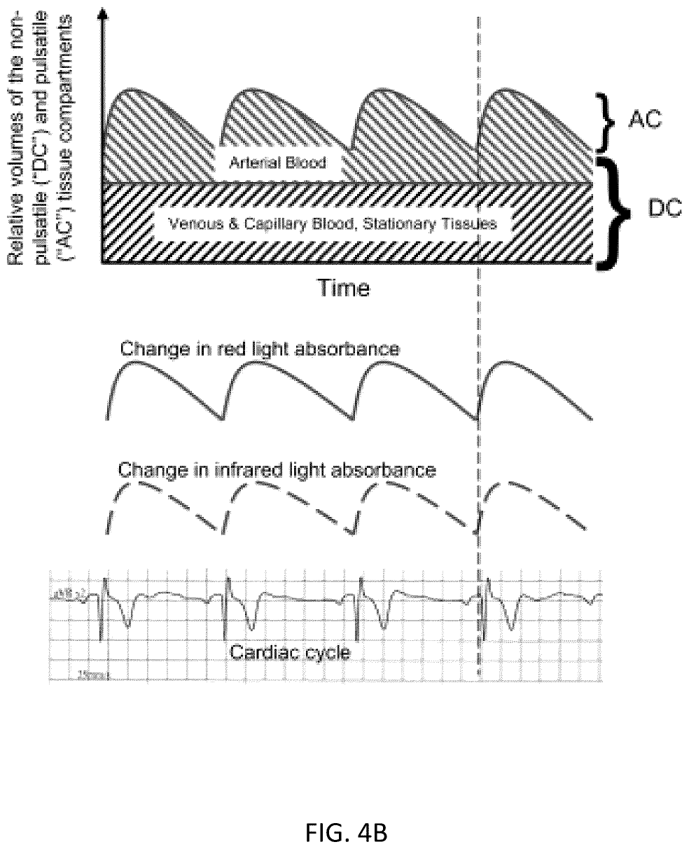

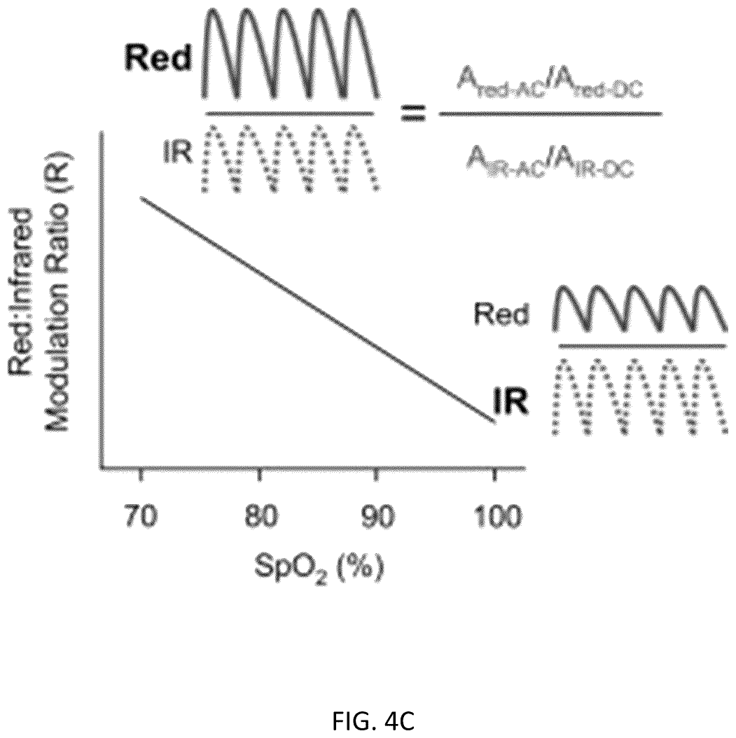

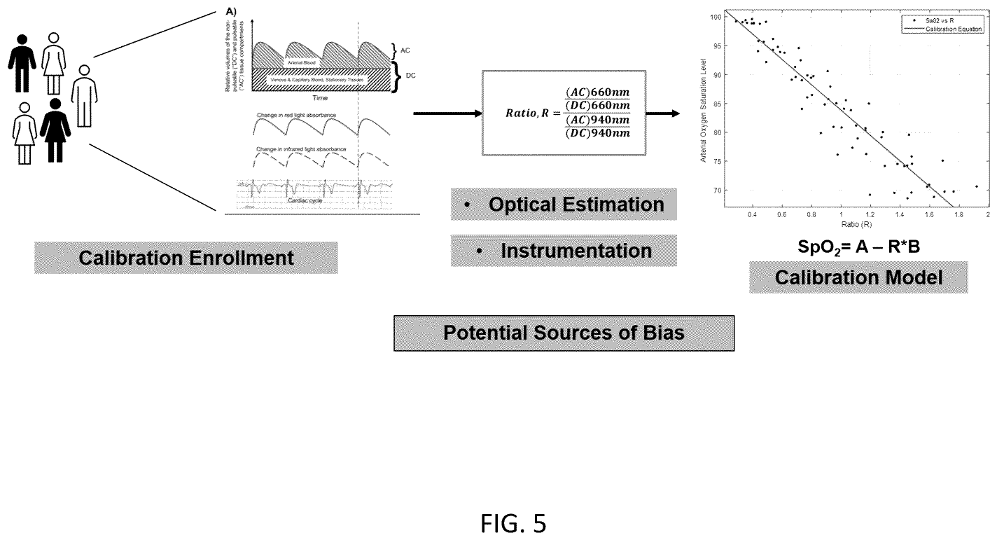

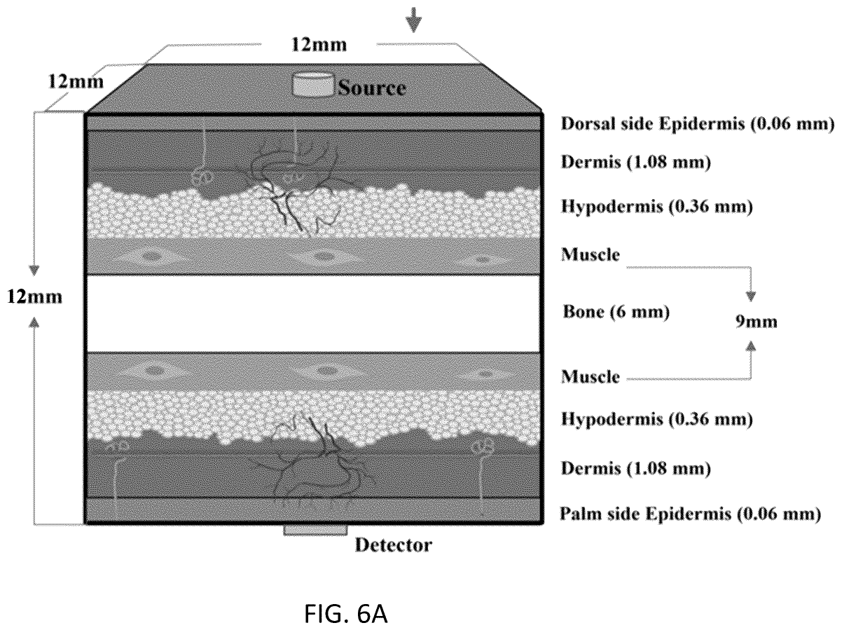

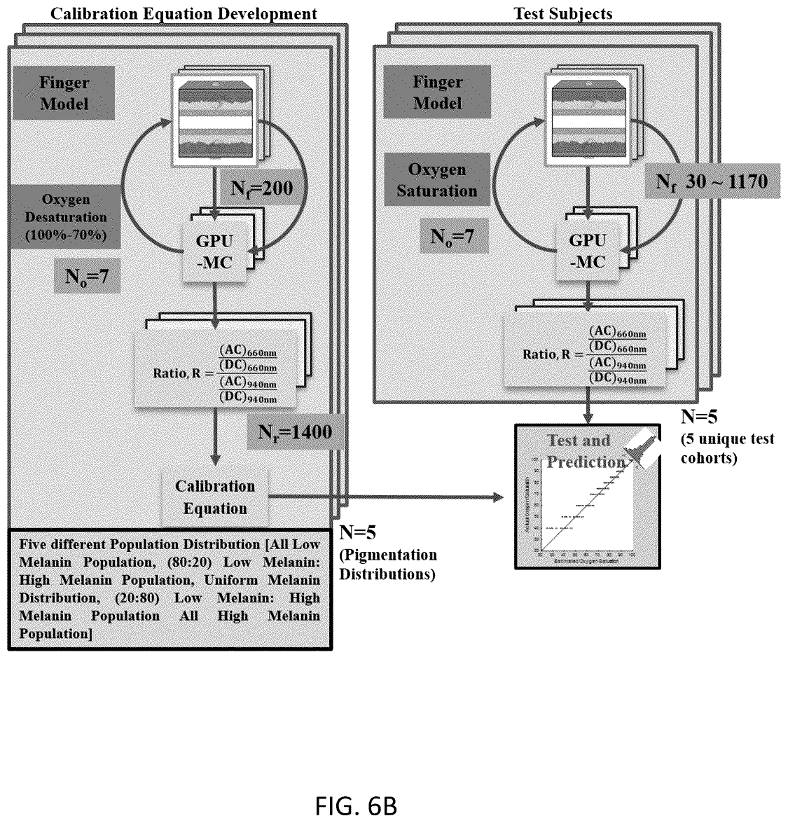

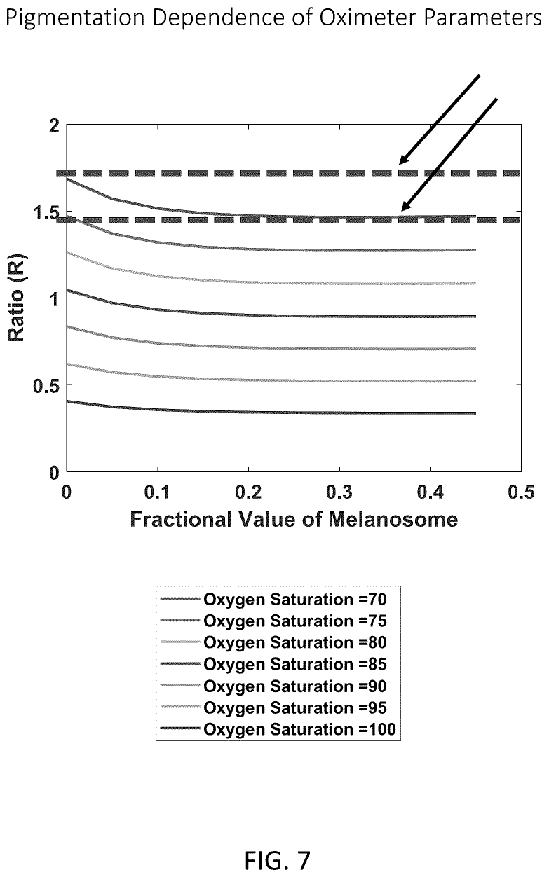

Referring now to FIG. 6A, shown is a model finger with an exemplary aspect of the invention showing an emitter (source) placed across from the detector. In various embodiments, each of the emitters may comprise light emitting diodes (LEDs) as arrays/matrices, liquid crystal displays (LCDs), organic light emitting diodes (OLEDs), or any combination thereof. The signals or optical/infrared signals emanating from the emitters may include a first emitting signal within the optical spectrum such as a red light which may be at or about 632-660 nm 2 and a second emitting signal that may be infrared (IR) at or around 940 nm 2 or may range from 905-940 nm 2. For each of the one or more emitters, the pulse oximeter may include one or more detectors which may be exemplary photodetectors, wherein each of the detectors is capable of receiving DC and/or AC components. The detectors may measure the signals, infrared signals, and/or optical signals through or reflected by the tissue as waveforms that may be periodic or nonperiodic. This may be referred to as photoplethysmography (PPG) which may be based on (i) a non-pulsatile ‘DC’ component driven by absorption from non-pulsatile tissue chromophores like melanin and venous blood volume and (ii) a pulsatile ‘AC’ component driven by absorption from time-varying arteriolar blood as shown in FIGS. 4-5.

Based on the measured signature by the one or more detectors, the processor of the oximeter may calculate the ratio of AC to DC components at red and infrared wavelengths, which defines the modulation ratio R, where R has an inverse linear relationship with blood oxygenation and may be defined as:

$$R = \frac{{AC}_{{λ}_{1}} / {DC}_{{λ}_{1}}}{{AC}_{{λ}_{2}} / {DC}_{{λ}_{2}}} \qquad Equation 1.$$

where λ₁ may be based on the optical range and λ₂ may be based on the infrared range.

λ₁ may be within the range of red light and/or may have a wavelength from 632 to 660 nm as discussed above. Similarly, λ₂ may be within the infrared band and/or may have a wavelength at or around 940 nm or may range from 905-940 nm λ as discussed above. By way of example, R may be calculated as follows:

$$R = \frac{{AC}_{660} / {DC}_{660}}{{AC}_{940} / {DC}_{940}} \qquad Equation 1.1$$

In one aspect of the invention, R may be modified to account for tissues of the subject that affect the DC component(s) of the resulting waveform(s). Such examples include but are not limited to non-pulsatile tissue chromophores like melanin and venous blood volume.

In another aspect of the invention, AC or DC component(s) of the resulting waveform(s) may be used. For example, when using one or more wavelengths from visible to short wave infrared, sources of biases or error (in the case of lipid content or hydration) status may be considered by using transmitted and/or reflected light.

In one aspect of the invention, the processor of the oximeter may calculate a derived index based on the DC component of the PPG signals corresponding to the two oximeters wavelengths (EQ 2.0).

$$Index = {log}_{10} \frac{{DC}_{{λ}_{2}}}{{DC}_{{λ}_{1}}} \qquad Equation 2.$$

where λ₁ may be based on the optical range and λ₂ may be based on the infrared range.

As discussed above, λ₁ may be within the range of red light and/or may have a wavelength from 632 to 660 nm. Similarly, λ₂ may be within the infrared band and may have a wavelength at or around 940 nm or may range from 905-940 nm λ.

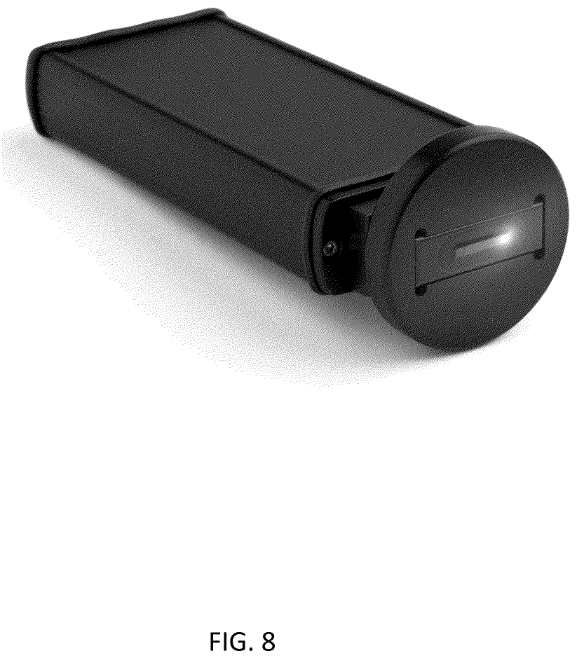

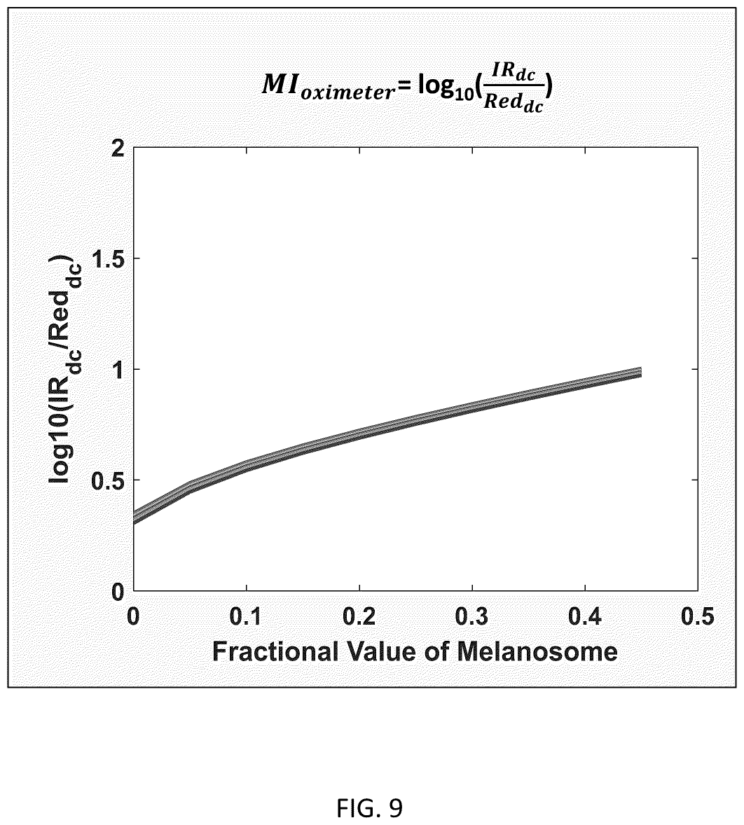

In the case of melanin, the processor of the oximeter may calculate an alternative derived melanin index (MIₒₓᵢₘₑₜₑᵣ). The parameter MIₒₓᵢₘₑₜₑᵣ is based on the optical melanin index (MI) obtained by conventionally Melanometers (as shown in FIG. 8) that is an additional component or hardware component that estimates melanin content in the skin based on the ratio of infrared and red-light intensity (see Treesirichod, A. et al., 2014. Indian Journal of Dermatology).

In one embodiment, the DC intensity is instead extracted from the pulse plethysmograph (PPG) signals corresponding to the two oximeter wavelengths are used in order to calculate MIₒₓᵢₘₑₜₑᵣ.

$${MI}_{oximeter} = {log}_{10} \frac{{DC}_{{λ}_{2}}}{{DC}_{{λ}_{1}}} \qquad Equation 2.1$$

As discussed above, λ₁ may be within the range of red light and/or may have a wavelength from 632 to 660 nm. Similarly, λ₂ may be within the infrared band and may have a wavelength at or around 940 nm or may range from 905-940 nm λ. By way of example, MIₒₓᵢₘₑₜₑᵣ may be calculate as follows:

$${MI}_{oximeter} = {log}_{10} \frac{{DC}_{940}}{{DC}_{660}} \qquad Equation 2.2$$

In one embodiment, when accounting for exemplary non-pulsatile tissue chromophores, the processor may calculate a modified R (R_{mod}) which is shown below. It can be appreciated that x and y, which are chosen empirically, are based on an optimization function cost function may be min. (gradient (R_{mod})).

R modified is calculated with the following equation:

$${R}_{m o d} = {R}^{x} {Index}^{y} \qquad Equation 3.$$

In an alternative embodiment, when accounting for melanin, the processor may calculate a modified R (R_{mod}) to create a pigmentation independent ratio by the following equation:

$${R}_{m o d} = {R}^{x} {MI}_{oximeter}^{y} \qquad Equation 3.1$$

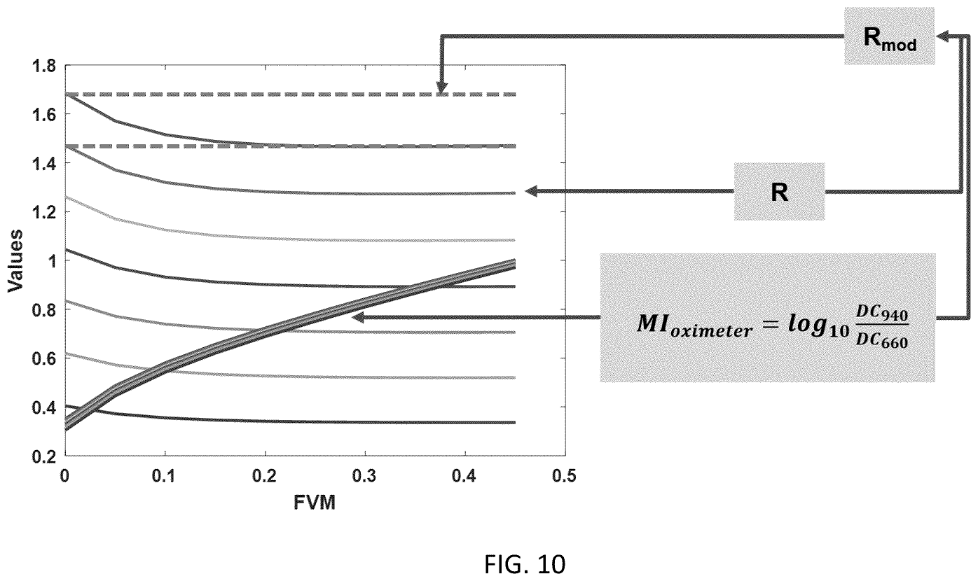

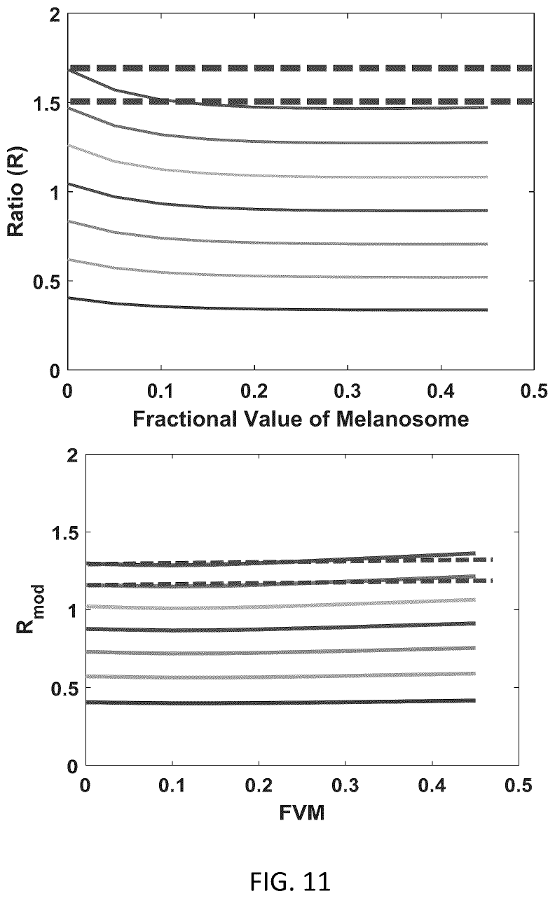

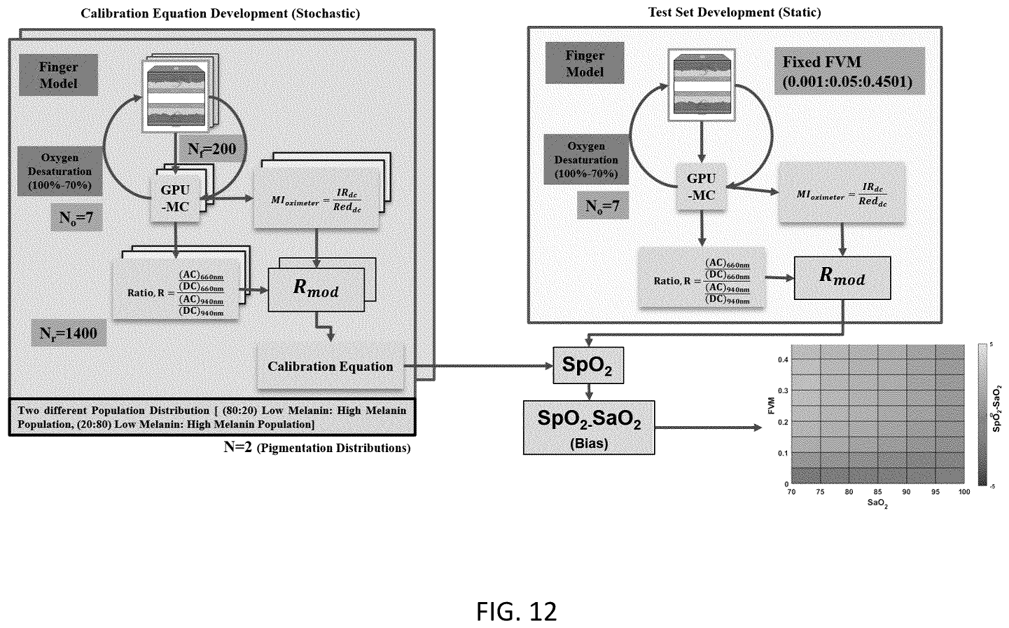

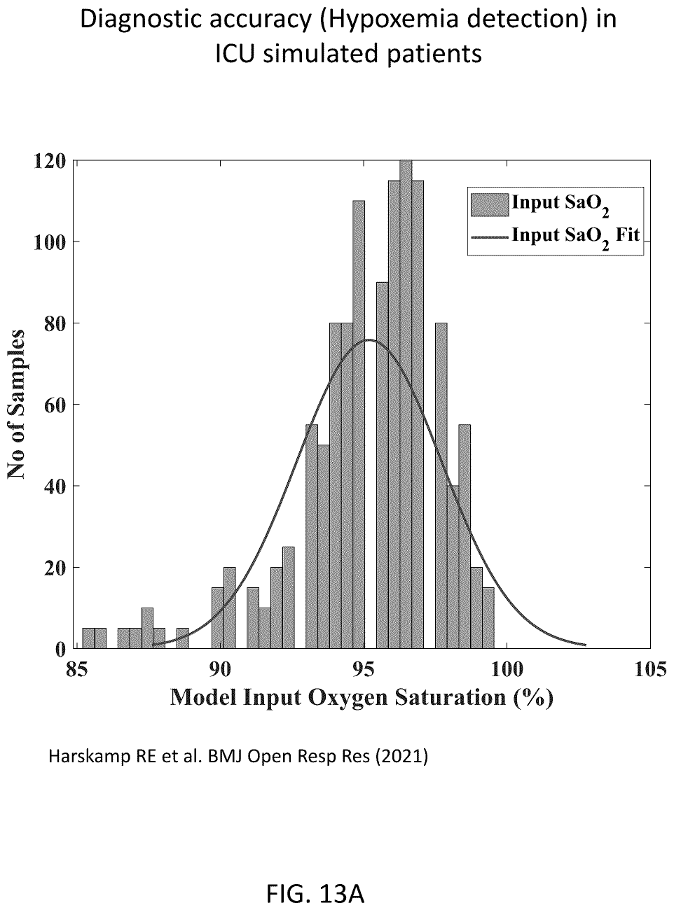

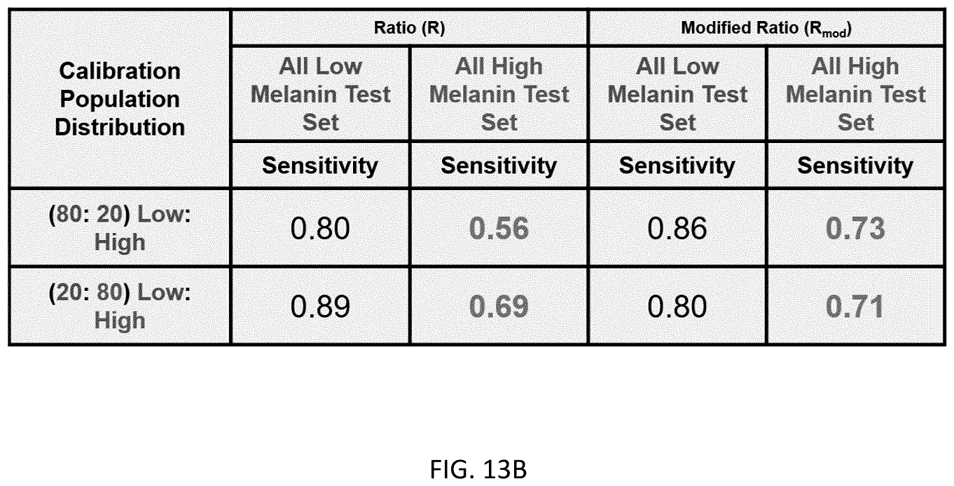

In one aspect of the invention, it can be appreciated that R vs. skin pigmentation curve shows opposite curvature to MIₒₓᵢₘₑₜₑᵣ vs. skin pigmentation curve as depicted in FIG. 10. Thus, a combination of these two opposite curvatures will generate a modified R_{mod} which would be independent of the skin pigmentation as shown in FIG. 10. This is advantageous as no additional hardware such as a Melanometer would be required.

In another aspect of the invention, one or more sensors may be configured to use more than two wavelengths. This process may be referred to as multi-wavelength pulse oximetry. This aspect of the invention may use variations of the equations above for calculation of the oximeter parameters. However, it can be appreciated by the skilled artisan that using multiple wavelengths from the same emitter or more than one emitter would reduce bias and error in multi-wavelength oximeters. In various embodiments, the oximeter may generate multiple signals with multiple IR wavelengths from a single emitter or may generate similar signals with multiple respective IR emitters for each wavelength. In various embodiments, the oximeter may generate optical and/or redlight emission with multiple signatures with multiple wavelengths from a signal emitter or may generate similar signals with multiple respective optical emitters for each of the multiple wavelengths.

In some aspects of the present invention, software executing the instructions provided herein may be stored on a non-transitory computer-readable medium, wherein the software performs some or all of the steps of the present invention when executed on a processor.

Aspects of the invention relate to algorithms executed in computer software. Though certain embodiments may be described as written in particular programming languages, or executed on particular operating systems or computing platforms, it is understood that the system and method of the present invention is not limited to any particular computing language, platform, or combination thereof. Software executing the algorithms described herein may be written in any programming language known in the art, compiled or interpreted, including but not limited to C, C++, C#, Objective-C, Java, JavaScript, MATLAB, Python, PHP, Perl, Ruby, or Visual Basic. It is further understood that elements of the present invention may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

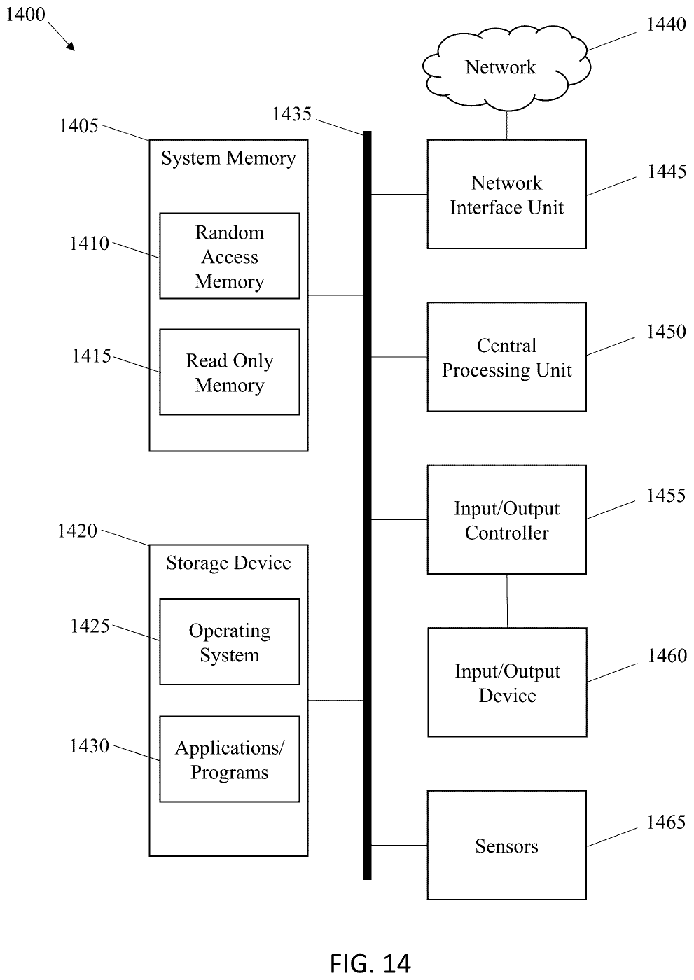

FIG. 14 and the following discussion are intended to provide a brief, general description of a suitable computing environment in which the invention may be implemented. While the invention is described above in the general context of program modules that execute in conjunction with an application program that runs on an operating system on a computer or may run on an embedded system, those skilled in the art will recognize that the invention may also be implemented in combination with other program modules.

Generally, program modules include routines, programs, components, data structures, and other types of structures that perform particular tasks or implement particular abstract data types. Moreover, those skilled in the art will appreciate that the invention may be practiced with other computer system configurations, including hand-held devices, multiprocessor systems, microprocessor-based or programmable consumer electronics, minicomputers, mainframe computers, and the like. The invention may also be linked through a communications network.

FIG. 14 depicts an illustrative computer architecture for a computer 1400 for practicing the various embodiments of the invention. The computer architecture shown in FIG. 14 illustrates a conventional computing device, including a central processing unit 1450 (“CPU”), a system memory 1405, including a random access memory 1410 (“RAM”) and a read-only memory (“ROM”) 1415, and a system bus 1435 that couples the system memory 1405 to the CPU 1450. A basic input/output system containing the basic routines that help to transfer information between elements within the computer, such as during startup, is stored in the ROM 1415. The computer 1400 further includes a storage device 1420 for storing an operating system 1425, application/program 1465, and data.

The storage device 1420 is connected to the CPU 1450 through a storage controller (not shown) connected to the bus 1435. The storage device 1420 and its associated computer-readable media provide non-volatile storage for the computer 1400. Although the description of computer-readable media contained herein refers to a storage device, such as a hard disk or CD-ROM drive, it should be appreciated by those skilled in the art that computer-readable media can be any available media that can be accessed by the computer 1400.

By way of example, and not to be limiting, computer-readable media may comprise computer storage media. Computer storage media includes volatile and non-volatile, removable and non-removable media implemented in any method or technology for storage of information such as computer-readable instructions, data structures, program modules or other data. Computer storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

The computing device 1400 may also include an input/output controller 1455 for receiving and processing input from a number of input/output devices 1460, including a keyboard, a mouse, a touchscreen, a camera, a microphone, a controller, a joystick, or other type of input device. Similarly, the input/output controller 1455 may provide output to a display screen, a printer, a speaker, or other type of output device. The computer 1400 can connect to the input/output device 1460 via a wired connection including, but not limited to, fiber optic, Ethernet, or copper wire or wireless means including, but not limited to, Wi-Fi, Bluetooth, Near-Field Communication (NFC), infrared, or other suitable wired or wireless connections.

As mentioned briefly above, a number of program modules and data files may be stored in the storage device 1420 and/or RAM 1410 of the computing device 1400, including an operating system 1425 suitable for controlling the operation of a networked computer. The storage device 1420 and RAM 1410 may also store one or more applications/programs 1465. In particular, the storage device 1420 and RAM 1410 may store an application/program 1465 for providing a variety of functionalities to a user. For instance, the application/program 1465 may comprise many types of programs such as a word processing application, a spreadsheet application, a desktop publishing application, a database application, a gaming application, internet browsing application, electronic mail application, messaging application, and the like. According to an embodiment of the present invention, the application/program 1465 comprises a multiple functionality software application for providing word processing functionality, slide presentation functionality, spreadsheet functionality, database functionality and the like.

### Experimental Examples

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples specifically point out exemplary embodiments of the present invention and are not to be construed as limiting in any way the remainder of the disclosure.

### In Silico Validation

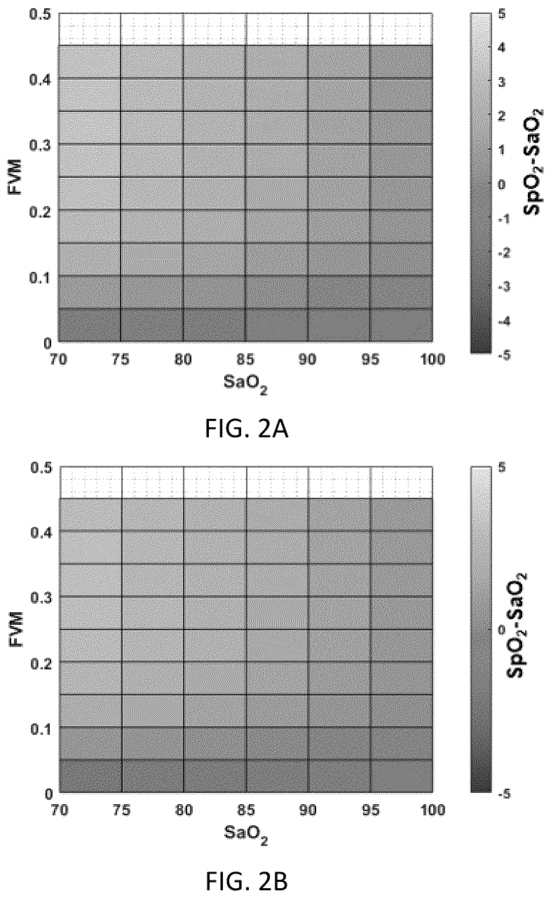

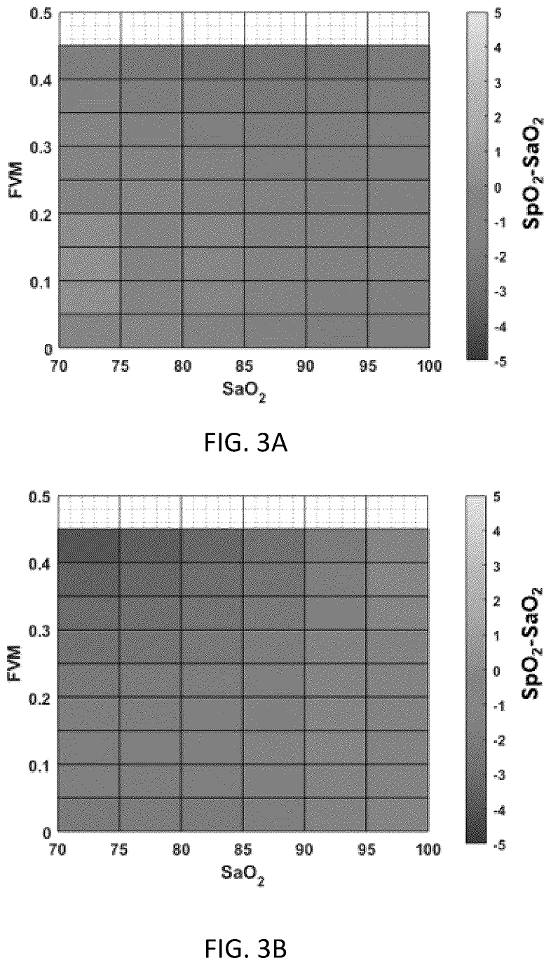

An oximeter ratio was customized using the parameters which are typically estimated in current two-wavelength pulse oximetry. The approaches stated here have been evaluated using computational simulations using Monte-Carlo modeling to simulate signals produced by finger-clip style pulse oximetry. The Monte-Carlo (MC) simulations of optical signals were produced by a transmittance-based pulse oximeter at two wavelengths (660 nm, 940 nm) as described in Ries et al. (see Ries, A. L. et al., 1989, CHEST). For both the oximeter parameter, R, and modified oximeter ratios R_{mod}, separate calibration studies were simulated with two different calibration enrollment demographics ([80:20] low:high pigmentation as shown in FIG. 2A and FIG. 3A and [50:50] low:high pigmentation as shown in FIG. 2B and FIG. 3B).

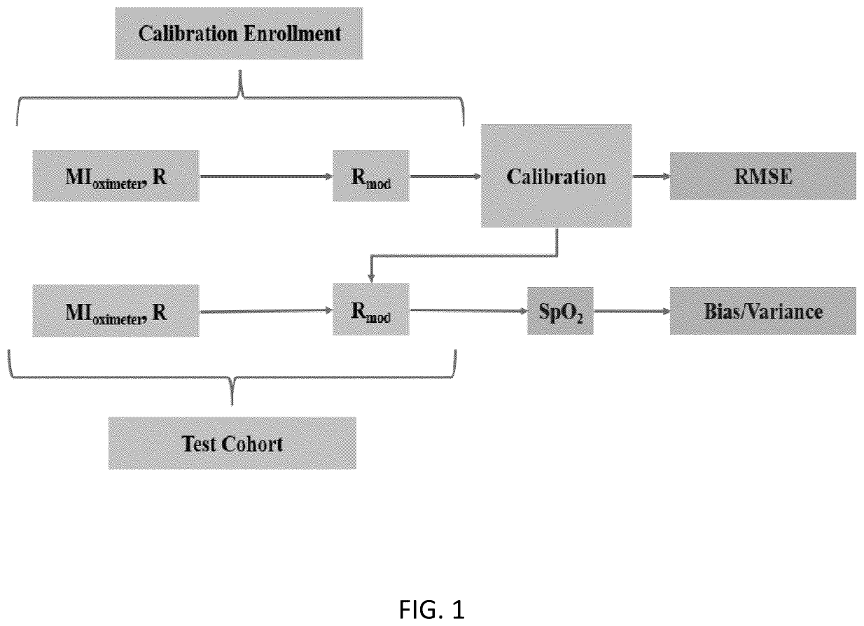

Estimation of SpO₂ is performed through an empirically determined calibration equation as seen in FIG. 1. Shown below is a typical calibration equation:

$${SpO}_{2} = 110 - 25 * R \qquad Equation 4$$

For a given subject, these models took as inputs melanin fraction and oxygen saturation and output predicted AC and DC red and infrared signals. A large data set of AC and DC red and infrared signals can thus be generated by running the model with input melanin fraction and oxygen saturation values that span a wide physiologically relevant range. As predicted from Equation EQ3.1, there is a strong linear relationship between the pulse-oximeter signal derived melanin index and the input melanin volume fraction which is maintained independent of blood oxygenation.

Specifically, the simulation comprised an initial cohort of subjects with known melanin fraction and systemic oxygenation. In each subject at each level of oxygenation, traditional modulation ratio R and derived melanin index MI new are determined, and R_{mod} is calculated with varying values for exponents x and y. Calibration equation parameters that minimize root mean square error (RMSE) between predicted SpO₂ and known blood oxygenation were then determined.

A test cohort of subjects was simulated with known melanin fraction and blood oxygenation (FIG. 1), and the bias between the predicted SpO₂ and known blood oxygenation was determined. Calibration simulations were performed with two different study populations. In one simulation the study population comprised of 20% of subjects having darkly pigmented skin and 80% of subjects having lightly pigmented skin (aligning with current minimum FDA requirements for oximeter validation studies) as depicted in FIG. 2A and FIG. 3A. In a second simulation there was an equal number of lightly pigmented and darkly pigmented subjects as depicted in FIG. 2B and FIG. 3B. The RMSE of the calibration equations calculated from these different study populations using R and different definitions of R_{mod} (based on x, y values).

Values x and y were set to 1 and yielded calibration curves with RMSE bit higher than the conventional approach. However, setting values x=0.8 and y=0.15 reduced the RMSE below the maximum threshold, resulting in calibration curves that would be deemed acceptable by current standards. These x and y values were selected from a pattern search optimization conducted in MATLAB. Next, the calibration curves derived were applied from both R and R_{mod} (x=0.8, y=0.15) to two test cohorts of subjects, one cohort with 80:20 lightly: darkly pigmented subjects, and the other cohort 50:50 light: darkly pigmented subjects. In each subject, the different calibration curves were used to provide an oximeter-based estimate of oxygenation (SpO₂) and subtracting arterial oxygen saturation (SaO₂) yielded device bias (SpO₂—SaO₂).

These simulated calibration enrollments were generated by stochastically sampling the epidermis and dermis optical properties. To simulate low melanin subjects, fractional volume of melanosome was considered to be less than 0.1, whereas, for high melanin subjects, fractional volume of melanosome distributions was greater than 0.1. For calibration studies, 200 different simulated subjects were generated with 7 different oxygenation levels ranging from 70% to 100%. The simulations were carried out using MCXLAB software (MCX for MATLAB) (see Fang, Q. et al., 2009, Opt. Express). After simulations, using estimated R and MIₒₓᵢₘₑₜₑᵣ, R_{mod} was calculated. To estimate the power factors x and y, an optimization algorithm was used. The optimization algorithm was constructed such that the gradient of the modified oximeter ration would be minimized, which would translate to pigmentation independence of the new oximeter ratio.

The accuracy of R and R_{mod} was tested on ten new simulated subjects having fractional volume melanosome (FVM) in the range of 0.001 to 0.450.

The x and y in EQ3.1 were evaluated based on an optimization algorithm (implemented in MATLAB) minimizing the pigmentation dependence of R_{mod}. One representative set of x, y values were selected as 0.8 and 0.15 respectively, to demonstrate potential improvements in pigmentation bias.

Referring now to FIG. 2A and FIG. 2B, shown is a visualization of device bias (color bar ranging from −5 blue to 5 yellow) as a function of arterial blood oxygenation SaO₂ and melanin volume fraction (FVM) where SpO₂ is determined using conventional modulation ratio R. Whether the test cohort is 80:20 lightly:darkly pigmented or 50:50 lightly:darkly pigmented, FIG. 2A and FIG. 2B clearly shows that oximeter derived SpO₂ overestimation of arterial oxygenation SaO₂ is more pronounced in the setting of hypoxia with increased skin pigmentation. This recapitulated findings in the literature of a pulse oximeter overestimation bias facing hypoxic patients with pigmented skin.

The simulated results indicated that when the oximeter ratio R is used, over-estimation bias increases with both increasing pigmentation (FVM) and decreasing arterial oxygen saturation levels (SaO₂) (FIG. 2A and FIG. 2B). Near the hypoxemia threshold (SaO₂<=88%), bias is greater than 0 which would support the idea of occult hypoxia in clinical settings relying upon oximeters.

Referring now to FIGS. 3A and 3B, shown is the same visualization as FIGS. 2A and 2B but the figures use R_{mod} (with x=0.8 and y=0.15) rather than conventional R to estimate SpO₂. In both population cohorts the pigmentation-associated device error that was present in FIG. 3 was significantly reduced if not eliminated. Thus, the use of modified R_{mod} reduces the magnitude of this pigmentation associated device overestimation, particularly in the setting of hypoxia (SpO₂<=88%) where accurate and timely clinical decision making is crucial. These Monte Carlo simulations indicated the calculation of a new oximeter parameter, R_{mod}, reduces over-estimation bias or the pigmentation bias. In addition, a combination of enrollment population and modified ratio, R_{mod} could eventually be helpful to make the pulse oximeter less susceptible to over-estimation and error. Using the modified ratio, reduced the over-estimation bias from 1.36% to −0.01% in the test cohort, when modification applied to the calibration generated from (80:20) low:high Melanin calibration enrollment suggests that over-estimation bias and overall error could be reduced using the modified oximeter ratio.

The computational simulations indicated modifying the calculation of the oximeter ratio using only optical signals inherent to existing pulse oximeters can reduce the pigmentation dependence of pulse oximeters. Put another way, the simulations supported the assertion that the existing pulse oximeter signal can be used to extract an estimate of skin pigmentation without the need for additional sensors or hardware.

### REFERENCES

Chan, E. D., Chan, M. M. & Chan, M. M. Pulse oximetry: Understanding its basic principles facilitates appreciation of its limitations. Respir. Medicine 107, 789-799, DOI: https://doi.org/10.1016/j.rmed.2013.02.004
Ralston, A. C., Webb, R. K., Runciman, W. B., 1991. Potential errors in pulse oximetry. Anaesthesia 46, 202-206. https://doi.org/10.1111/j.1365-2044.1991.tb09410.x
Fine, J., Branan, K. L., Rodriguez, A. J., Boonya-ananta, T., Ajmal, Ramella-Roman, J. C., McShane, M. J., Coté, G. L., 2021. Sources of Inaccuracy in Photoplethysmography for Continuous Cardiovascular Monitoring. Biosensors 11. https://doi.org/10.3390/bios11040126
Jubran, A., 2015. Pulse oximetry. Critical Care 19, 272. https://doi.org/10.1186/s13054-015-0984-8
Ries, A. L., Prewitt, L. M., Johnson, J. J., 1989. Skin Color and Ear Oximetry. CHEST 96, 287-290. https://doi.org/10.1378/chest.96.2.287
Sjoding, M. W., Dickson, R. P., Iwashyna, T. J., Gay, S. E., Valley, T. S., 2020. Racial Bias in Pulse Oximetry Measurement. New England Journal of Medicine. https://doi.org/10.1056/NEJMc2029240
Fawzy, A. et al. Racial and Ethnic Discrepancy in Pulse Oximetry and Delayed Identification of Treatment Eligibility Among Patients With COVID-19. JAMA Intern. Medicine 182, 730-738, DOI: 10.1001/jamainternmed.2022.1906 (2022).
Valbuena, V. S. M., Seelye, S., Sjoding, M. W., Valley, T. S., Dickson, R. P., Gay, S. E., Claar, D., Prescott, H. C., Iwashyna, T. J., 2022. Racial bias and reproducibility in pulse oximetry among medical and surgical inpatients in general care in the Veterans Health Administration 2013-19: multicenter, retrospective cohort study. BMJ 378, e069775. https://doi.org/10.1136/bmj-2021-069775
Boonya-ananta, T. et al. Synthetic photoplethysmography (ppg) of the radial artery through parallelized monte carlo and its correlation to body mass index (bmi). Sci. Reports 11, 2570, DOI: 10.1038/s41598-021-82124-4 (2021). Number: 1 Publisher: Nature Publishing Group.
Arefin, M. S., Dumont, A. P. & Patil, C. A. Monte carlo based simulations of racial bias in pulse oximetry. In Hwang, J. & Vargas, G. (eds.) Design and Quality for Biomedical Technologies XV, vol. 11951, 8-12, DOI: 10.1117/12.2610483 (SPIE, 2022). Backup Publisher: International Society for Optics and Photonics.
NIH announces winners of 2022 DEBUT Challenge [WWW Document], n.d.. National Institute of Biomedical Imaging and Bioengineering. URL https://www.nibib.nih.gov/news-events/newsroom/nih-announces-winners-2022-debut-challenge (accessed 7.7.23).
Baek, H. J., Shin, J., Cho, J., 2018. The Effect of Optical Crosstalk on Accuracy of Reflectance-Type Pulse Oximeter for Mobile Healthcare. Journal of Healthcare Engineering 2018, e3521738. https://doi.org/10.1155/2018/3521738
Okunlola, O. E., Lipnick, M. S., Batchelder, P. B., Bernstein, M., Feiner, J. R., Bickler, P. E., 2022. Pulse Oximeter Performance, Racial Inequity, and the Work Ahead. Respiratory Care 67, 252-257. https://doi.org/10.4187/respcare.09795
Keller, M. D., Harrison-Smith, B., Patil, C., Arefin, M. S., 2022. Skin colour affects the accuracy of medical oxygen sensors. Nature 610, 449-451. https://doi.org/10.1038/d41586-022-03161-1
Treesirichod, A., Chansakulporn, S., Wattanapan, P., 2014. Correlation between skin color evaluation by skin color scale chart and narrowband reflectance spectrophotometer. Indian Journal of Dermatology 59, 339.
Fang, Q., Boas, D. A., 2009. Monte Carlo Simulation of Photon Migration in 3D Turbid Media Accelerated by Graphics Processing Units. Opt. Express, OE 17, 20178-20190. https://doi.org/10.1364/OE.17.020178

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention.

What is claimed is:

1. A device for measuring blood oxygen saturation comprising:
at least two emitters comprising:
one or more IR emitters configured to:
emit one or more first signals at one or more respective first wavelengths, wherein the one or more first signals are able to be absorbed in part by oxyhemoglobin;
one or more second emitters configured to:
emit one or more second signals at one or more respective second wavelengths, wherein the one or more second signals are able to be absorbed in part in by deoxyhemoglobin;
one or more detectors configured to receive (i) one or more first waveforms corresponding to the one or more first signals and (ii) one or more second wave forms corresponding to the one or more second signals, wherein the one or more first and second waveforms each comprise AC and DC components; and
one or more processors operatively coupled to the one or more detectors, wherein the processor is configured to calculate an oximeter ratio (R) based on the respective AC and DC components of the one or more first and second waveforms.

2. The device of claim 1, wherein the one or more IR emitters emit the one or more first wavelengths from about 850 nm to 1000 nm.

3. The device of claim 1, wherein the one or more second emitters emit the one or more second wavelengths from about 600 nm to 750 nm.

4. The device of claim 3, wherein the one or more second emitters emit the second wavelength from about 632 nm to 660 nm.

5. The device of claim 1, wherein the one or more processors calculate the oximeter ratio to correct for one or more of:lipid content, hydration status, non-pulsatile tissue, chromophores, melanin, and venous blood volume.

6. The device of claim 1, wherein the device is capable of being placed on a forehead, nose, foot, ear, or toe of a patient.

7. The device of claim 1, wherein the one or more IR emitters consist of one IR emitter to emit one first signal at one first wavelength, wherein the one first signal is able to be absorbed in part by oxyhemoglobin.

8. The device of claim 1, wherein the one or more second emitters consist of one second emitter to emit one second signal at one second wavelength, wherein the one second signal is able to be absorbed in part by deoxyhemoglobin.

9. The device of claim 1:
wherein the one or more IR emitters consist of one IR emitter configured to:
emit a plurality of first signals at a respective plurality of first wavelengths; and
wherein the one or more second emitters consist of one second emitter configured to:
emit a plurality of second signals at a respective plurality of second wavelengths.

10. The device of claim 1:
wherein the one or more IR emitters consist of one IR emitter to emit one first signal at one first wavelength, wherein the one first signal is able to be absorbed in part by oxyhemoglobin; and
wherein the one or more second emitters consist of one second emitter to emit one second signal at one second wavelength, wherein the one second signal is able to be absorbed in part by deoxyhemoglobin;
wherein the one or more first and second waveforms consist of one first waveform and one second waveform;
one or more detectors are configured to receive (i) the one first waveform corresponding to the one first signal and (ii) the one second wave form corresponding to the one second signal, wherein the one first waveform and one second waveform each comprise AC and DC components, respectively; and
wherein the calculated oximeter ratio (R) is based on the respective AC and DC components of the one first waveform and one second waveforms.

11. The device of claim 10, wherein the oximeter ratio (R) is modified to correct for melanin using a calculated index (MI) to create a modified oximeter ratio (R).

12. The device of claim 11, wherein the calculated index (MI) is based on the respective DC components of the one first waveform and one second waveform.

13. The device of claim 12, wherein the calculated melanin index (MI) is based on a logarithmic function.

14. The device of claim 13, wherein the calculated index is used to account for blood volume.

15. The device of claim 10, wherein the oximeter ratio (R) is modified to correct for melanin using a calculated melanin index (MI) to create a modified oximeter ratio (R).

16. The device of claim 15, wherein the calculated melanin index (MI) is based on the respective DC components of the one first waveform and one second waveform.

17. The device of claim 16, wherein the calculated melanin index (MI) is based on a logarithmic function and used to account for melanin.

18. The device of claim 1, wherein the one or more detectors comprise one or more photodetectors.

19. A device for measuring blood oxygen saturation comprising:
one first emitter configured to:
emit more than one first signal at more than one respective first wavelengths, wherein the more than one first signal is able to be absorbed in part by oxyhemoglobin;
one second emitter configured to:
emit more than one second signal at more than one respective second wavelengths, wherein the more than one second signal is able to be absorbed in part in by deoxyhemoglobin;
one or more detectors configured to receive (i) more than one first waveforms corresponding to the more than one first signal and (ii) more than one second wave forms corresponding to the more than one second signal, wherein the more than one first and second waveforms each comprise AC and DC components; and
a processor operatively coupled to the one or more detectors, wherein the processor is configured to calculate a modified oximeter ratio (R) to improve measurement quality based on the respective AC and DC components of the more than one first and second waveforms.

20. A device for measuring blood oxygen saturation using only a first and second emitter comprising:
the first emitter configured to emit a first signal at a first wavelength;
the second emitter configured to a second signal at a second wavelength;
one or more detectors configured to receive a first and second waveform corresponding to the first and second signal, respectively, wherein the first and second waveforms each comprise AC and DC components; and
one or more processors operatively coupled to the one or more detectors, wherein the processor is configured to calculate a modified oximeter ratio (R) based on the AC and DC components of the first and second signals, wherein R is pigmentation independent.
